(19) United States
(12) Patent Application Publication
(10) Pub. No.: US 2006/0008539 A1
(43) Pub. Date: Jan. 12, 2006

(54) Coating-type antimicrobial composition, antimicrobial coating film, filter, and electric air-quality conditioning equipment

(75) Inventor: Toshikazu Tomioka, Osaka (JP)

Correspondence Address: MCDERMOTT WILL & EMERY LLP, 600 13TH STREET, N.W., WASHINGTON, DC, 20005-3096, US

(73) Assignee: MATSUSHITA ELECTRIC INDUSTRIAL CO., LTD.

(21) Appl. No.: 11/171,199

(22) Filed: Jul. 1, 2005

(30) Foreign Application Priority Data
Jul. 9, 2004 (JP) 2004-203734
Jul. 9, 2004 (JP) 2004-203745

(51) Int. Cl. A61K 36/53

(52) U.S. Cl. 424/725

(57) ABSTRACT

A coating-type antimicrobial composition contains at least a water extract of a plant body and a paint resin and/or a coating solvent. The plant body is, e.g., genus Eucalyptus, genus Kaemferia, genus Isodon, genus Scutellaria, genus Usnea, genus Pistacia, and genus Senecio.

### CROSS REFERENCE TO RELATED APPLICATIONS

This application claims priority under 35 U.S.C. §119 on Patent Application No. 2004-203734 filed in Japan on Jul. 9, 2004 and Patent Application No. 2004-203745 filed in Japan on Jul. 9, 2004, the entire contents of which are hereby incorporated by reference.

### BACKGROUND OF THE INVENTION

1. Technical Field

The present invention relates to a technology for suppressing proliferation of microorganisms that are present in the living environment or for killing microorganisms. The present invention also relates to a technology that is effective to improve cleanliness of the living environment, to prevent degradation of construction materials, to prevent allergies of residents, and the like.

2. Background Art

When mold grows in construction materials that are used in the living environment, appearance is spoiled and strength of the construction materials is reduced. Moreover, mold can damage residents' health as an allergen. Recently, consumers increasingly prefer to purchase antibacterial daily-use products such as writing tools (pens, pencils, and the like). Antibacterial treatment is therefore demand for various daily-use materials. In order to meet such a demand, various antibacterial compositions for forming an antibacterial coating film on a material surface have been developed (e.g., Japanese Laid-Open Patent Publication No. 2000-154339).

### SUMMARY OF THE INVENTION

Since consumers are becoming more and more natural products-oriented in recent years, traditionally used natural materials have been desired to be used for antimicrobial coating films. It is an object of the present invention to provide a highly safe antimicrobial coating film which has little effect on human bodies and which is effective against microorganisms (especially fungi) commonly inhabiting the living environment.

The present invention uses a water extract of at least one kind of plant body selected from the group consisting of: branches and leaves of a plant of family Futomomo (Japanese name) (Myrtaceae), genus Yukarinoki (Japanese name) (Eucalyptus); a rhizome of a plant of family Shoga (Japanese name) (Zingiberaceae), genus Ban-ukon (Japanese name) (Kaempferia); a whole plant body of an aboveground portion of a plant of family Shiso (Japanese name) (Laminaceae (Labiatae)), genus Isodon (Japanese name) (Isodon); a rhizome of a plant of family Shiso (Japanese name) (Laminaceae (Labiatae)), genus Tatsunamiso (Japanese name) (Scutellaria); a whole plant body of a plant of family Saruogase (Japanese name) (Usneaceae), genus Saruogase (Japanese name) (Usnea); bark, branches and leaves of a plant of family Urushi (Japanese name) (Anacardiaceae), genus Ranshinboku (Japanese name) (Pistacia); and a whole plant body of a plant of family Kiku (Japanese name) (Compositae), genus Sawagiku (Japanese name) (Senecio).

Water extracts of branches and leaves of plants of family Myrtaceae, genus Eucalyptus and water extracts of rhizomes of plants of family Zingiberaceae, genus Kaempferia have antibacterial action against Gram-negative bacteria such as Escherichia coli. Water extracts of whole plant bodies of aboveground portions of plants of family Laminaceae (Labiatae), genus Isodon; water extracts of rhizomes of plants of family Laminaceae (Labiatae), genus Scutellaria; water extracts of whole plant bodies of plants of family Usneaceae, genus Usnea; water extracts of bark, branches and leaves of plants of family Anacardiaceae, genus Pistacia; and water extracts of whole plant bodies of plants of family Compositae, genus Senecio have antibacterial action against Gram-positive bacteria such as Staphylococcus aureus.

The present invention also uses an organic-solvent extract of at least one kind of plant body selected from the group consisting of a whole plant body of an aboveground portion of a plant of family Laminaceae (Labiatae), genus Isodon; a rhizome of a plant of family Labiatae, genus Scutellaria; a whole plant body of a plant of family Usneaceae, genus Usnea; bark, branches and leaves of a plant of family Anacardiaceae, genus Pistacia; and a rhizome of a plant of family Tade (Japanese name) (Polygonaceae), genus Daio (Japanese name) (Rheum). These organic-solvent extracts have antibacterial action against Gram-positive bacteria such as Staphylococcus aureus.

Due to traditionally recognized pharmacological action, plant bodies that are used in the present invention have been used for treatment of diseases as Chinese herb medicine, or the action of the plant bodies have been passed down as folk medicine. Hereinafter, specific examples of these plant bodies and their usage will be shown.

Plants of family Myrtaceae, genus Eucalyptus include Eucalyptus globulus Labill., Eucalyptus smithii R. T. Baker, Eucalyptus citriodora Hook., and the like. For example, Yukarinoki (Japanese name), Eucalyptus globulus Labill., from China is used for treatment of neuralgia as external medicine or for treatment of bronchitis by vapor inhalation.

Plants of family Laminaceae (Labiatae), genus Isodon include Isodon eriocalyx (Dunn) Kuds, Isodon long itubus, Isodon shikokianus var. intermedius, Isodon umbrosus, Isodon umbrosus var. excisinflexus, and the like. Manshu-hikiokoshi (Japanese name), Isodon eriocalyx (Dunn) Kuds, is used for cold, swollen or sore throat, gastritis, hepatitis, tonsillitis, mastitis, initial cancer, menoschesis, bruise, arthralgia, insect bite, and snakebite.

Plants of family Laminaceae (Labiatae), genus Scutellaria include Scutellaria baicalensis Georgi, Scutellaria cavaleriei, Scutellaria hebeclada, Scutellaria mairei, Scutellaria taguetii, Scutellaria thieretti, Scutellaria vaniotiana, Scutellaria veronicifolia, and the like. Koganeyanagi (Japanese name), Scutellaria baicalensis Georgi, is used for prevention of scarlatina and for treatment of acute enterocolitis, diarrhea, acute hepatitis, and chronic hepatitis.

Plants of family Zingiberaceae, genus Kaempferia include Ban-ukon (Japanese name) (Kaempferia galanga L.) and the like. Kaempferia palanga L. is used for treatment of stomachache, vomiting, diarrhea, and the like as decoction and for treatment of toothache as external medicine.

Plants of family Usneaceae, genus Usnea include Usnea longissima Ach., Usnea pangiana, Usnea rubescens, Usnea diffracta, and the like. Nagasaruogase (Japanese name), Usnea longissima Ach., is used for treatment of pulmonary tuberculosis, various kinds of inflammations, and the like as decoction and is externally used for treatment of various kinds of ulcers as catapasm. In recent years, however, this plant has been increasingly imported as incense ingredients and has been less and less used as medicine.

Plants of family Anacardiaceae, genus Pistacia include Ranshinboku (Japanese name), Pistacia weinmannifolia J. Poisson ex Franch. Pistacia weinmannifolia J. Poisson ex Franch is said to be used for symptoms such as diarrhea, itching skin, and boils, but is not used as a medicine in Japan.

Plants of family Compositae, genus Senecio include Senecio scandens Buch.-Ham, Senecio madagascariensis Poiret, Senecio rowlevanus, Senecio macroplossus, Senecio nemorensis, Senecio anteuphorbium, Senecio plaberrimus, Senecio keniodendron, Senecio platyphylloides, Senecio hybridus, Senecio fremontii, Senecio inteprifolius subsp. fauriei, Senecio vulzaris, and the like. Taikin-giku (Japanese name), Senecio scandens Buch.-Ham, is used for upper respiratory tract infection, tonsillitis, sore throat, pneumonia, conjunctivitis, dysentery, enterocolitis, appendicitis, acute lymphangitis, erysipelas, eczema, allergic dernatitis, and the like, but is not used as a medicine in Japan.

Plants of family Polygonaceae, genus Rheum include Rheum officinale Baill., Rheum palmatum L., Rheum tanguticum Maxim., Rheum coreanum Nakai, Rheum rhaponticum Linn., Rheum undulatum L., and the like. Daio (Japanese name), Rheum officinale Baill., is used as a large-intestine purgative, an antiphlogistic stomachic, and the like.

As described above, plant bodies that are used in the present invention have been used mainly for treatment of diseases and the like, and have been used merely as symptomatic treatment. There are only a small number of research reports about antimicrobial action (e.g., antibacterial action) of components of these plant bodies. For example, Japanese Laid-Open Patent Publication No. 11-80012 discloses that polar-organic-solvent extracts of branches and leaves of plants of family Myrtaceae, genus Eucalyptus are effective for Trichophyton, acne bacteria, or MRSA (methicillin resistant Staphylococcus aureus). As described below, however, water extracts of the present invention have higher antibacterial action to Gram-negative bacteria than the polar-organic-solvent extracts disclosed in the above publication.

The present invention provides a coating-type antimicrobial composition and an antimicrobial coating film which are described below. In the following description, the coating-type antimicrobial composition and the antimicrobial coating film are sometimes simply referred to as a “composition” and a “coating film,” respectively.

A coating-type antimicrobial composition according to a first aspect of the present invention contains at least: a water extract of at least one kind of plant body selected from the group consisting of branches and leaves of Eucalyptus globulus Labill. of family Myrtaceae, a rhizome of Kaempferia galangal L. of family Zingiberaceae, a whole plant body of an aboveground portion of Isodon eriocalyx (Dunn) Kuds of family Laminaceae (Labiatae), a rhizome of Scutellaria baicalensis Georgi of family Laminaceae (Labiatae), a whole plant body of Usnea longissima Ach. of family Usneaceae, bark, branches and leaves of Pistacia weinmannifolia J. Poisson ex Franch of family Anacardiaceae, and a whole plant body of Senecio scandens Buch.-Ham. of family Compositae; and a paint resin and/or a coating solvent.

A coating-type antimicrobial composition according to a second aspect of the present invention contains at least: an organic-solvent extract of at least one kind of plant body selected from the group consisting of a whole plant body of an aboveground portion of Isodon eriocalyx (Dunn) Kuds of family Laminaceae (Labiatae), a rhizome of Scutellaria baicalensis Georgi of family Laminaceae (Labiatae), a whole plant body of Usnea lonpissima Ach. of family Usneaceae, bark, branches and leaves of Pistacia weinmannifolia J. Poisson ex Franch of family Anacardiaceae, and a rhizome of Rheum officinale Baill. of family Polygonaceae; and a paint resin and/or a coating solvent.

An antimicrobial coating film according to a third aspect of the present invention contains at least a water extract of at least one kind of plant body selected from the group consisting of: branches and leaves of Eucalyptus globulus Labill. of family Myrtaceae; a rhizome of Kaempferia galanga L. of family Zingiberaceae; a whole plant body of an aboveground portion of Isodon eriocalvx (Dunn) Kuds of family Laminaceae (Labiatae); a rhizome of Scutellaria baicalensis Georgi of family Laminaceae (Labiatae); a whole plant body of Usnea longissima Ach. of family Usneaceae; bark, branches and leaves of Pistacia weinmannifolia J. Poisson ex Franch of family Anacardiaceae; and a whole plant body of Senecio scandens Buch.-Ham. of family Compositae. This antimicrobial coating film typically further contains a paint resin.

An antimicrobial coating film according to a fourth aspect of the present invention contains at least an organic-solvent extract of at least one kind of plant body selected from the group consisting of: a whole plant body of an aboveground portion of Isodon eriocalyx (Dunn) Kuds of family Laminaceae (Labiatae); a rhizome of Scutellaria baicalensis Georgi of family Laminaceae (Labiatae); a whole plant body of Usnea longissima Ach. of family Usneaceae; bark, branches and leaves of Pistacia weinmannifolia J. Poisson ex Franch of family Anacardiaceae; and a rhizome of Rheum officinale Baill. of family Polygonaceae. This antimicrobial coating film typically further contains a paint resin.

In the specification, a “plant body” includes not only the whole plant (whole plant body) but a part of a plant such as branches, leaves, rhizomes, and bark. “Microorganisms” include bacteria, filamentous fungi, yeast, myxomycetes, single-cell algae, protozoa, and viruses. A “coating-type antimicrobial composition” is a coating-type composition for suppressing proliferation of microorganisms or for killing microorganisms. Examples of the coating-type antimicrobial composition include an antibacterial paint and an antibacterial spray for providing antibacterial action to industrial products by applying the antibacterial paint or spray to the industrial products. An “antimicrobial coating film” is a coating film for suppressing proliferation of microorganisms or for killing microorganisms. For example, the antimicrobial coating film is formed by applying an antibacterial paint or an antibacterial spray.

A “water extract” is an extract of active ingredients from a plant body by steam distillation, immersion, or the like. An “organic-solvent extract” is an extract of active ingredients from a plant body by immersing the plant body in an organic solvent (which includes a mixed solvent of water and an organic solvent). Note that a water extract and an organic-solvent extract are sometimes collectively referred to as a “plant body extract.”

A “coating solvent” refers to a solvent for dissolving or dispersing a plant body extract and a paint resin (binder) therein. Preferably, the coating solvent is at least one solvent selected from the group consisting of water, methyl alcohol, ethyl alcohol, isopropyl alcohol, and acetone.

An organic-solvent extract is an extract obtained using an organic solvent or a mixed solvent of water and an organic solvent. Examples of an organic solvent include ethyl alcohol, methyl alcohol, acetone, chloroform, benzene, methylene chloride, and the like. Examples of a mixed solvent of water and an organic solvent include a mixed solvent of water and acetone and a mixed solvent of water and ethyl alcohol. The mixing weight ratio of water to an organic solvent is 5 to 95 parts by weight of water to 95 to 5 parts by weight of an organic solvent, preferably 15 to 85 parts by weight of water to 85 to 15 parts by weight of an organic solvent, and more preferably, 25 to 75 parts by weight of water to 75 to 25 parts by weight of an organic solvent.

Active ingredients can be extracted from a plant body by a commonly used method. For example, extraction may be conducted by immersing an extraction part of a plant in a solvent over an extended period of time, by putting a plant in a solvent, heating the solvent at a temperature equal to or lower than the boiling point of the solvent while stirring, and then filtering the solvent to obtain an extract. It is desirable to prepare an extract by concentrating an extraction solution by an evaporator or a spray dry method.

A composition of the present invention may be a water paint such as a water-soluble resin paint and a synthetic resin emulsion paint. A coating film of the present invention may contain, as a paint resin, a resin included in a water paint such as a water-soluble resin paint and a synthetic resin emulsion paint. A water-soluble resin paint is a paint that uses a water-soluble resin forming a water-soluble colloid as a paint resin (hereinafter, sometimes referred to as a “paint binder”). Examples of a water-soluble resin forming a water-soluble colloid include carboxymethylcellulose and polyvinyl alcohol. A synthetic resin emulsion paint is a paint prepared by dispersing in water a synthetic resin emulsion obtained by emulsion polymerization or the like as a paint binder. Commonly used synthetic resins are acrylic resin, polyester resin, epoxy resin, urethane resin, silicone resin, fluorine resin, phenol resin, alkyd resin, aminoalkyd resin, urea resin, unsaturated resin, vinyl resin, and the like. Examples of a highly hydrophilic paint resin include polyvinyl alcohol, carboxymethylcellulose, epoxy resin, urethane resin, and polyester resin.

Preferably, a paint contains 1 to 30 parts by weight of a paint resin for 70 to 99 parts by weight of a plant body extract, and more preferably, 1 to 20 parts by weight of a paint resin for 80 to 99 parts by weight of a plant body extract, based on a solid content.

A coating film of the present invention is typically formed by applying an antimicrobial paint to a base material and drying the applied antimicrobial paint. The antimicrobial paint includes at least a plant body extract of the present invention, a paint resin of the present invention, and a coating solvent for dispersing the plant body extract and the paint resin of the present invention therein. Note that a coating solvent refers to a solvent for forming a coating film, and is herein distinguished from a solvent for extracting active ingredients from a plant body.

An antimicrobial paint, one form of a composition of the present invention, can be prepared by mixing a dispersion in which at least one kind of plant body extract is dispersed and a paint resin and/or a coating solvent. Alternatively, the antimicrobial paint may be prepared by dispersing at least one kind of plant body extract in a liquid containing a paint resin and/or a coating solvent.

A coating solvent for dispersing a plant body extract therein is selected based on solubility of the plant body extract and compatibility with the paint resin. More specifically, one kind of solvent or a mixed solvent (a mixture of a plurality of kinds of solvents) selected from the group consisting of water, methyl alcohol, ethyl alcohol, isopropyl alcohol, and acetone can be preferably used as a coating solvent for dispersing a plant body extract therein.

A solvent for dispersing a plant body extract therein and a solvent contained in a paint are typically of the same kind, but may be of different kinds. For example, a solvent for dispersing a plant body extract therein may be water, and a solvent contained in a paint may be a mixed solvent of water and ethyl alcohol.

A coating film may contain 1 to 500 parts by weight of a plant body extract, preferably 1 to 100 parts by weight of a plant body extract, and more preferably 1 to 50 parts by weight of a plant body extract, for 100 parts by weight of a solid matter of a paint resin.

A coating film of the present invention does not have to contain a paint resin. In other words, in the specification, an “antimicrobial paint” includes a paint having a plant body extract and a coating solvent mixed and dispersed therein.

A composition and a coating film of the present invention may further contain an inorganic carrier. Preferably, the inorganic carrier is formed mainly from a porous inorganic oxide such as silica gel. The inorganic carrier may be particulate powder having silanol groups at its surface. In terms of adsorptivity of an extract to the organic carrier, it is desirable that the inorganic carrier have a specific surface area of at least 300 m²/g, and preferably at least 500 m²/g. In terms of unevenness of a coating film surface, it is desirable that the inorganic carrier have a grain size of at most 50 μm, and preferably a grain size of 1 μm to 30 μm.

Note that the specific surface area can be measured according to a Brunauer-Emmett-Teller (BET) method described in “The journal of the American Chemical Society,” Vol. 60, page 309, February 1938 and a BET method based on measurement of gas adsorption. The grain size of the particulate powder can be measured by an optical microscope, a scanning electron microscope, or a sedimentation method.

The inorganic carrier may be particulate powder formed from an intercalation compound. Examples of the intercalation compound include hydrotalcite compounds and smectite compounds. Hydrotalcite compounds are shown by general formula: [M²⁺₁₋ₓM³⁺ₓ(OH)₂]ₓ₊[Aⁿ⁻_{x/n}.yH₂O]ₓ₋.

(where M²⁺ indicates bivalent metal ions such as magnesium, nickel, cobalt, manganese, and zinc, M³⁺ indicates trivalent metal ions such as aluminum, iron, manganese, and chromium, and Aⁿ⁻ indicates n-valent anions such as hydroxyl group, chlorine, nitrate group, carbonate group, and sulfate group.)

Hydrotalcite compounds include trigonal carbonate minerals shown by general formula: A₆B₂(CO₃)(OH)₁₆.4H₂O (where A=Mg, Ni, and B=Al, Cr³⁺, Fe³⁺, Mn²⁺, Co³⁺). An example of such carbonate minerals is hydrotalcite shown by Mg₆AI₂(CO₃)(OH)₁₆.4H₂O. Other examples of such carbonate minerals are comblainite, desautelsite, iowaite, pyroaurite, reevesite, stichtite, takovite, and the like.

Smectite compounds are monoclinic silicate minerals shown by general formula: X_{0.3}Y₂₋₃Z₄O₁₀(OH)₂.nH₂O (where X (exchangeable ion)=Ca/2, Li, Na; Y=Al, Cr³⁺, Cu²⁺, Fe²⁺, Fe³⁺, Li, Mg, Ni, Zn; and Z=Al, Si). An example of such silicate minerals is montmorillonite shown by (Na, Ca)0.3(Al, Mg)₂Si₄O₁₀(OH)₂.nH₂O. Other examples of such silicate minerals are aliettite, beidellite, hectorite, nontronite, saponite, sauconite, stevensite, swinefordite, volkonskoite, yakhontovite, and the like.

The grain size of the inorganic carrier is preferably in the range of 1 μm to 2,000 μm, and more preferably in the range of 10 μm to 1,000 μm.

When a composition of the present invention contains a paint resin, a solid matter of a paint after a coating process preferably contains 10 to 50 parts by weight of an inorganic carrier and 49 to 89 parts by weight of a plant body extract for 1 to 30 parts by weight of the paint resin.

A coating film of the present invention may contain 1 to 30 parts by weight of a paint resin, 10 to 50 parts by weight of an inorganic carrier, and 49 to 89 parts by weight of an extract.

The composition and the coating film of the present invention may contain another antibacterial agent or disinfectant, a surfactant, a repellent, an efficacy enhancer, an insect repellent, an attractant, a deodorizer, an ultraviolet (UV)-blocking agent (including a UV absorber and an agent for preventing UV-induced degradation), an antioxidant, a thickener, a stabilizer, a brightener, a pigment, a dye, a filler, and the like. The composition and the coating film of the present invention may contain a synthetic antifungal agent.

A composition and a coating film which contain a plant body extract typically contain an extract of ingredients other than active ingredients, too. An extract of active ingredients and an extract of ingredients other than the active ingredients are both likely to become resources for microorganisms. Therefore, decay may proceed. Decayed extracts may not only degrade the active ingredients but also attract pests.

In order to prevent decay of plant body extracts, the composition and the coating film of the present invention desirably contain a silver-based antibacterial agent and/or a natural antibacterial agent. The silver-based antibacterial agent may contain a carrier holding silver complexes or silver ions. An example of the carrier holding silver complexes is silica gel holding thiosulphato silver complexes (trade name: Amenitop, a product of Matsushita Electric Industrial Co., Ltd.). Examples of the carrier holding silver ions are zeolite holding silver ions, phosphates holding silver ions, and glass holding silver ions. These carriers may have at least one kind of a plant body extract adsorbed therein.

Catechin, an extract of Japanese horseradish (Japanese name: Wasabi) (Eutrema iaponica of family Aburana (Japanese name) (family Cruciferae)), an extract of Mosochiku (Japanese name) (Phyllostachys pubescens of family Ine (Japanese name) (family Gramineae)), or the like can be used as a natural antibacterial agent. Alternatively, a synthetic compound of an active ingredient contained in a natural antibacterial agent may be used. For example, a synthetic compound of allylisothiocyanate contained in a Wasabi extract may be used.

Preferably, a composition and a coating film of the present invention contain 0.1 to 50 parts by weight of a silver-based antibacterial agent or a natural antibacterial agent for 100 parts by weight of a total solid matter. As mentioned above, the composition and the coating film of the present invention may contain a synthetic antifungal agent in order to obtain antifungal action. This improves an anti-decomposition property of the antimicrobial coating film against true fungi.

The present invention also provides an industrial product according to fifth to eighth aspects of the present invention described below. An industrial product according to a fifth aspect of the present invention includes an electric air-quality conditioning equipment having an air filter attached thereto. Examples of the electric air-quality conditioning equipment include an air cleaner, an air conditioner, a humidifier, and a vacuum cleaner. The air filter is formed from woven fabric, non-woven fabric, or a combination of woven fabric and non-woven fabric. The antimicrobial coating film of the present invention is formed at least at intersections of fibers.

An industrial product according to a sixth aspect of the present invention includes a sheet having the antimicrobial coating film of the present invention printed or applied to a surface. The antimicrobial coating film is formed by printing or applying an antimicrobial paint to the sheet surface with various patterns (e.g., a dot pattern, a stripe pattern, or a matrix pattern). The surface area of the antimicrobial coating film is at least 50%, and preferably at least 90%, of the sheet surface area. Examples of a method for printing or applying the antimicrobial paint include a spray coating method, a screen printing method, a roll coater method, a gravure method, a relief printing method, a dip brazing method, and the like.

Typically, the sheet is made of paper, cloth, metal, or synthetic resin, and has a single-layer or multi-layer structure. The sheet is provided at a prescribed position of the industrial product. For example, the sheet is provided at positions that are frequently in contact with users' fingers and at damp positions where bacteria can proliferate. Examples of such positions are control panels such as a touch panel, input or control-equipment interface panels, pushbutton panels, and under the floor. The sheet having the antimicrobial coating film of the present invention printed or applied to its surface may be wallpaper to be attached or fixed to a wall surface.

An industrial product according to a seventh aspect of the present invention is an electric equipment having the antimicrobial coating film of the present invention formed on a housing, a base material of a part, or a circuit board (typically, a printed circuit board). The antimicrobial coating film is formed by printing or applying an antimicrobial paint to one or both surfaces of the housing, the base material of the part, or the circuit board or by impregnating the housing, the base material of the part, or the circuit board with the antimicrobial paint. Examples of the electric equipment include electrical cooking equipments, home electrical appliances, security equipments, communication equipments, office equipments, toiletry equipments, and the like. Specific examples of the electrical equipment include a refrigerator, a wine cellar, a freezer, a microwave oven, an electric thermo pot, a mixer, a juicer, a food processor, a hot plate, an electric grill pot, a fish roaster, a cooking heater, a mochi/dough maker, a water cooler/heater, an electrical water heater, an alkaline ionized water apparatus, a rice cooker, a coffee maker, a coffee mill, a home bread maker, a water purifier, a mineral water apparatus, a system kitchen, a dish washer, a dish drier, a garbage disposer, a washing machine, a clothes dryer, a vacuum cleaner, an ionized rinse water maker, a futon drier, a polisher, a bath buzzer, an electric bucket-type washer, a shredder, a dehumidifier, a dehumidifying drier, an air cleaner, a shower toilet seat, an air conditioner, a kerosene stove, an electric stove, an electric fan, a humidifier, an electric blanket, a hot-water room heater, a kerosene far-infrared heater, an electric air heater, an oil heater, a kerosene fan heater, a kerosene air heater, a radiation heater, a gas water heater, an electric water heater, a ventilating fan, an underfloor drier, a heat pump water heater, a shaver, a hair depilator/remover, a hair drier, an electric toothbrush, a massaging chair, a lighting equipment, a wiring equipment, an alarm terminal, a flame detector, a smoke detector, a pencil sharpener, a desk cleaner, a television, a videocassette recorder, a video camera, an audio equipment, a DVD (Digital Versatile Disc) player, a personal computer, a peripheral equipment for a personal computer, a facsimile, an electronic musical instrument, a telephone, a cell phone, and the like.

An industrial product according to an eighth aspect of the present invention includes a member having the antimicrobial coating film of the present invention formed thereon. The antimicrobial coating film is formed by printing or applying an antimicrobial paint to the member or by impregnating the member with the antimicrobial paint. The member is typically a base material made of paper, metal, or synthetic resin. The member may have any shape as long as the shape is suitable for an industrial product to which the member is to be mounted. For example, the antimicrobial coating film can be formed on a surface by applying an antimicrobial paint to a sheet, a tube, a molded article having a curved surface, or the like.

The industrial product according to the eighth aspect of the present invention includes various kinds of members and equipments. Examples of the industrial product include construction materials, stationery, clothing, accessories, home gardening materials, office equipments, interior members, exterior members, vending machines, auto parts, parts of electric products, traffic-related equipments, furniture, daily necessities, cooking equipments, medical products, and medical and welfare facilities.

Examples of the construction materials include wallpaper, an underfloor sheet, and a handrail. Examples of the stationery include a fountain pen, a mechanical pencil, a ballpoint pen, a pencil case, a document box, a carry case, a document holder, a plastic sheet (that is placed under a sheet of paper when writing), a mouse pad, a card folder, scissors, toys, baby goods, and musical instruments. Examples of the clothing include underwear, socks, a shirt, a towel, a bed sheet, a bedspread, sleepwear, a pillow, futon, futon wadding, a bed, and a mattress. Examples of the accessories include a belt, glasses, watch accessories, gloves, an apron, and false-teeth related equipments.

Examples of the home gardening materials include a multi film, victoria lawn, a greenhouse film, a fruit cover bag, a drawing string, and a support column. Examples of the office equipments include a file cabinet, an office supply cabinet, and a locker.

Examples of the interior members include a cupboard, an underfloor storage, an animal cage, curtains, and a blind. Examples of the exterior members include a waterproof sheet, a fence, and a window screen.

Examples of the auto parts include a steering wheel, a seat member, in-car cleaning products, a car air conditioner, a vehicle-mounted refrigerator, and a vehicle-mounted AV (Audio-Visual) equipment. Examples of the parts of electric products include parts that are used in the above electric equipments. Examples of the traffic-related equipments include a strap, an in-vehicle ETC (Electronic Toll Collection system) unit, an ETC card, and a train communication apparatus (an in-vehicle pay phone). Examples of the furniture include a cupboard and a bed.

Examples of the daily necessities include bath products, toilet articles, a toothbrush, hair care products, kitchenware, dinnerware, tableware, one-way containers, returnable bottles, and storage containers.

Examples of the cooking equipments include a rice cooker, a mixer, a refrigerator, a wine cellar, a serving tray, a rice bin, and a seasoning dispenser. Examples of the medical products include cosmetics, an adhesive plaster, contact lens care products, and a mask. Examples of the medical and welfare facilities include nursing care products, a cane, a bed, a wheel chair, bedding, a serving cart, and a toilet bowl.

### BRIEF DESCRIPTION OF THE DRAWINGS

FIG. 1 is a partial enlarged view of a pre-filter layer.

### DETAILED DESCRIPTION OF THE INVENTION

Hereinafter, embodiments of the present invention will be described. The following embodiments use at least one kind of plant body extract from the following plant body extracts: a water extract of branches and leaves of Eucalyptus globulus Labill. of family Myrtaceae; a water extract of a rhizome of Kaempferia galanga L. of family Zingiberaceae; a water extract of a whole plant body of an aboveground portion of Isodon eriocalvx (Dunn) Kuds of family Laminaceae (Labiatae); a water extract of a rhizome of Scutellaria baicalensis Georgi of family Laminaceae (Labiatae); a water extract of a whole plant body of Usnealongissima Ach. of family Usneaceae; a water extract of bark of Pistacia weinmannifolia J. Poisson ex Franch of family Anacardiaceae; a water extract of branches and leaves of Pistacia weinmannifolia J. Poisson ex Franch of family Anacardiaceae; a water extract of a whole plant body of Senecio scandens Buch.-Ham of family Compositae; an organic-solvent extract of a whole plant body of an aboveground portion of Isodon eriocalyx (Dunn) Kuds of family Laminaceae (Labiatae); an organic-solvent extract of a rhizome of Scutellaria baicalensis Georgi of family Laminaceae (Labiatae); an organic-solvent extract of a whole plant body of Usnealoniissima Ach. of family Usneaceae; an organic-solvent extract of bark of Pistacia weinmannifolia J. Poisson ex Franch of family Anacardiaceae; an organic-solvent extract of branches and leaves of Pistacia weinmannifolia J. Poisson ex Franch of family Anacardiaceae; and an organic-solvent extract of a rhizome of Rheum officinale Baill. of family Polygonaceae. Note that, in the following embodiments, a paint is used as a coating-type antimicrobial composition. A coating-type antimicrobial composition is herein referred to as an antimicrobial paint.

### First Embodiment

Antibacterial action of a water extract of branches and leaves of Eucalyptus globulus Labill. of family Myrtaceae from China, a method for producing an antimicrobial paint containing the water extract, and a method for producing an antimicrobial coating film will be briefly described below. Branches and leaves of Eucalyptus globulus Labill. of family Myrtaceae were picked and dried in the shade in preparation for extraction. A warm-water soluble material was obtained by water-extracting the branches and leaves of Eucalyptus globulus Labill., more specifically, by steam distilling the branches and leaves of Eucalyptus globulus Labill. A dark-brown sticky material was obtained by concentrating the warm-water soluble material with a rotary evaporator.

Commonly used extraction methods include a method for immersing an extraction part of a plant in an organic solvent over an extended period of time, and a method for heating a mixture of an extraction part of a plant and an organic solvent at a temperature equal to or lower than the boiling point of the organic solvent while stirring, and then filtering the resultant mixture to obtain an extract. However, water extraction produces an extract having high antibacterial action and a wide antibacterial spectrum.

Evaluation of antibacterial action includes evaluation of antibacterial action of extracts themselves and evaluation of antibacterial action of antimicrobial coating films produced for practical use by processing extracts. In the following description, antibacterial action of extracts themselves is evaluated and antibacterial action of antimicrobial coating films is then evaluated.

Respective antibacterial actions of a water extract of the first embodiment and a polar-organic-solvent extract disclosed in Japanese Laid-Open Patent Publication No. 11 -80012 were evaluated. The result is shown in Table 1 for comparison. Note that acetone, a ketone, was used as a polar organic solvent.

In evaluation of antibacterial action, Escherichia coli and Staphylococcus aureus were used as an example of Gram-negative bacteria and Gram-positive bacteria, respectively. Evaluation was conducted according to standard testing methods (the method of the Japanese Society of Chemotherapy and the method of the Society of Industrial-Technology for Antimicrobial Articles) and using minimum inhibitory concentration (MIC), a commonly used index of antibacterial action. Antibacterial action is evaluated by Minimum inhibitory concentration (MIC) for strains. A smaller MIC value indicates stronger antibacterial action. It is herein defined that a sample has antibacterial action when a MIC value is 1,600 or less (ppm).

TABLE 1
Comparison of Antibacterial Action (MIC) between organic-solvent
extract and water extract of various plant bodies

| Plant body | | | | |
|---|---|---|---|---|
| (Japanese name/ | Organic-solvent extract | | Water extract | |
| scientific name) and | Escherichia | Staphylococcus | Escherichia | Staphylococcus |
| bacteria species | coli | aureus | coli | aureus |
| Yukarinoki | 3200 | 200 | 800 | 400 |
| (Eucalyptus globulus Labill.) | | | | |
| Ban-ukon | 3200 | 3200 | 1600 | 1600 |
| (Kaempferia galangal L.) | | | | |
| Manshu-hikiokoshi | 3200 | 50 | 3200 | 400 |
| (Isodon eriocalyx (Dunn) Kuds) | | | | |
| Koganeyanagi | 3200 | 800 | 3200 | 800 |
| (Scutellaria baicalensis Georgi) | | | | |
| Nagasaruogase | 3200 | 25 | 3200 | 12.5 |
| (Usnea longissima Ach.) | | | | |
| Ranshinboku | 3200 | 1600 | 3200 | 800 |
| (Pistacia weinmannifolia J. | | | | |
| Poisson ex Franch) | | | | |
| Taikin-giku | 3200 | 3200 | 3200 | 1600 |
| (Senecio scandens Buch. -Ham) | | | | |
| Daio | 3200 | 400 | 3200 | 3200 |
| (Rheum officinale Baill.) | | | | |

As shown in Table 1, a water extract of Eucalyptus globulus Labill. of the first embodiment had antibacterial action against Gram-negative bacteria such as Escherichia coli and Gram-positive bacteria such as Staphylococcus aureus. An organic-solvent extract of Eucalyptus globulus Labill. had antibacterial action only against Staphylococcus aureus and did not have good or practical antibacterial action against Escherichia coli.

The result of Table 1 shows that extracts have different antibacterial spectra depending on the extraction methods. More specifically, no antibacterial effect against Gram-negative bacteria such as Escherichia coli was found in the organic-solvent extract disclosed in Japanese Laid-Open Patent Publication No. 11-80012 in terms of numerical evaluation such as MIC. However, the water extract was found to have a wide antibacterial spectrum and an antibacterial effect against Gram-negative bacteria as well.

Hereinafter, a method for producing an antimicrobial paint containing a water extract of the present embodiment will be described. First, 10 parts by weight (based on solid weight) of an aqueous acrylic resin emulsion (trade name: PAW aqueous screen ink, a product of Toyo Ink Mfg. Co., Ltd.) was prepared as a paint binder (paint resin). 1 part by weight of the water extract was mixed with the aqueous acrylic resin emulsion and well dispersed therein. An antimicrobial paint was thus prepared.

As shown below, an antimicrobial coating film was prepared by the following method using this antimicrobial paint, and antibacterial action of the antimicrobial coating film was evaluated.

1. The antimicrobial paint is thinly applied to a PET (polyethylene terephthalate) film. The coating amount of the antimicrobial paint to the PET film is about 0.1 g (before drying). Note that a PET film of 125 μm thick (trade name: Lumilar (U943), a product of Toray Industries, Inc.) was used in the present embodiment.

2. After the antimicrobial paint is applied, the PET film is air-dried at ordinary temperature. The PET film is then forced-dried for one hour at 60° C. with a drier in preparation for testing.

3. A microbial solution (0.4 ml) is dropped on the coating film. The coating film is then covered with a film and is let stand for 24 hours at 37° C. The microbial solution is then collected and cultured, and a viable cell count is measured. Note that the culture time was 48 hours for Staphylococcus aureus and 24 hours for Escherichia coli.

A test piece was produced in the same manner as that of the present embodiment except that the water extract was not used. The test piece thus produced was evaluated as a blank. Another test piece was produced in the same manner as that of the present embodiment by using an organic-solvent extract. Table 2 shows the evaluation test result of antibacterial action of the antimicrobial coating film of the present embodiment. Table 3 shows the evaluation test result of antibacterial action of the antimicrobial coating film produced with an organic-solvent extract.

Note that, the coating films having a MIC value of 3,200 or more (ppm) are not shown in Tables 2 and 3 due to their poor antibacterial action.

TABLE 2
Evaluation result of antibacterial action of coating films
containing water extracts of various plant bodies

| | | Staphylococcus aureus | | Escherichia coli | |
|---|---|---|---|---|---|
| Plant body | | | Viable cell | | Viable cell |
| (Japanese | | MIC | count | MIC | count |
| name) | Scientific name | (ppm) | (cfu/ml) | (ppm) | (cfu/ml) |
| Initial | — | | 7.1 × 10⁵ | | 7.8 × 10⁵ |
| cell count | | | | | |
| Blank | — | | 6.7 × 10⁷ | | 8.2 × 10⁷ |
| Yukarinoki | Eucalyptus globulus | 400 | <10 | 800 | <10 |
| | Labill. | | | | |
| Ban-ukon | Kaempferia galangal L. | 1600 | 2.3 × 10² | 1600 | 14 |
| Manshu- | Isodon eriocalyx (Dunn) | 400 | <10 | — | — |
| hikiokoshi | Kuds | | | | |
| Koganeyanagi | Scutellaria baicalensis | 800 | <10 | — | — |
| | Georgi | | | | |
| Nagasaruogase | Usnea longissima Ach. | 12.5 | <10 | — | — |
| Ranshinboku | Pistacia weinmannifolia J. | 800 | <10 | — | — |
| | Poisson ex Franch | | | | |
| Taikin-giku | Senecio scandens Buch. | 1600 | 2.8 × 10² | — | — |
| | -Ham. | | | | |

TABLE 3
Evaluation result of antibacterial action of coating films
containing organic-solvent extracts of various plant bodies

| | | Staphylococcus aureus | | Escherichia coli | |
|---|---|---|---|---|---|
| Plant body | | | Viable cell | | Viable cell |
| (Japanese | | MIC | count | MIC | count |
| name) | Scientific name | (ppm) | (cfu/ml) | (ppm) | (cfu/ml) |
| Initial | — | | 7.1 × 10⁵ | | 7.8 × 10⁵ |
| cell count | | | | | |
| Blank | — | | 6.7 × 10⁷ | | 8.2 × 10⁷ |
| Yukarinoki | Eucalyptus globulus Labill. | 200 | <10 | — | — |
| (reference) | | | | | |
| Manshu- | Isodon eriocalyx (Dunn) Kuds | 50 | <10 | — | — |
| hikiokoshi | | | | | |
| Koganeyanagi | Scutellaria baicalensis Georgi | 800 | <10 | — | — |
| Nagasaruogase | Usnea longissima Ach. | 25 | <10 | — | — |
| Ranshinboku | Pistacia weinmannifolia J. | 1600 | 3.5 × 10² | — | — |
| | Poisson ex Franch | | | | |
| Daio | Rheum officinale Baill. | 400 | <10 | — | — |

The result shows that an antimicrobial coating film containing a low MIC extract has high antibacterial action. More specifically, the antimicrobial coating film containing a water extract of Eucalyptus globulus Labill. had high antibacterial action against Gram-negative bacteria such as Escherichia coli (see Table 2). On the other hand, an antimicrobial coating film containing an organic-solvent extract of Eucalyptus globulus Labill. did not have good antibacterial action against Gram-negative bacteria (see Table 3).

Both the antimicrobial coating film containing a water extract of Eucalyptus globulus Labill. and the antimicrobial coating film containing an organic-solvent extract of Eucalyptus globulus Labill. had high antibacterial action against Gram-positive bacteria such as Staphylococcus aureus (see Tables 2 and 3). It was confirmed from the above result that the antimicrobial coating film of the present embodiment containing a water extract of Eucalyptus globulus Labill. had high antibacterial action against Gram-negative bacteria and Gram-positive bacteria.

In the above example, an aqueous acrylic resin emulsion was used to produce a coating film from the water extract. However, the same antibacterial action was obtained even when other resins were used as a paint resin. More specifically, the same antibacterial action as that obtained when an aqueous acrylic resin emulsion was used was obtained even when emulsions containing other polymers such as polyester resin, epoxy resin, urethane resin, silicone resin, or fluorine resin were used. Moreover, it was confirmed that practical antibacterial action as an antimicrobial coating film was obtained even when water-soluble resins such as polyvinyl alcohol and carboxymethylcellulose were used as a highly hydrophilic resin or binder.

In particular, since a plant body extract was produced by water extraction, the plant body extract is highly hydrophilic and has a high affinity with a water-based paint. The antimicrobial coating film of the present embodiment can therefore be produced by using a water extract that can be easily developed into a water-based paint. In other words, the antimicrobial coating film of the present embodiment can be produced without using organic solvents, which is advantageous in terms of a coating process and is highly industrially beneficial in terms of environmental pollution. Moreover, microorganisms live with water and fly as water evaporates. Therefore, antibacterial components in water extracts have a high affinity with water around microorganisms. Accordingly, the antimicrobial coating film of the present embodiment has a practical advantage in terms of the effect on microorganisms. In other words, antibacterial components in the antimicrobial coating film of the present embodiment are very likely to affect microorganisms.

### Second Embodiment

Antibacterial action of a water extract of a rhizome of Kaempferia palangal L. of family Zingiberaceae from China, a method for producing an antimicrobial paint containing the water extract, a method for producing an antimicrobial coating film, and antibacterial action of the antimicrobial coating film will be briefly described below. A rhizome of Kaempferia galangal L. of family Zingiberaceae was picked and dried in the shade in preparation for extraction. A warm-water soluble material was obtained by water-extracting the rhizome of Kaempferia galangal L., more specifically, by steam distilling the rhizome of Kaempferia palangal L. A light-brown liquid material was obtained by concentrating the warm-water soluble material with a rotary evaporator.

Respective antibacterial actions of a water extract of the second embodiment and an organic-solvent (acetone) extract were evaluated. The result is shown in Table 1 for comparison. Note that species of bacteria, a method for evaluating antibacterial action, and an index (MIC) which are used in the second embodiment are the same as those used in the first embodiment. It is herein defined that a sample has antibacterial action when a MIC value is 1,600 or less (ppm).

The result shows that the water extract of Kaempferia galangal L. of the present embodiment had antibacterial action against Gram-negative bacteria such as Escherichia coli and Gram-positive bacteria such as Staphylococcus aureus although the antibacterial action was slightly weaker than that of the water extract of Eucalyptus globulus Labill. of the first embodiment. However, the organic-solvent extract of Kaempferia galangal L. did not have antibacterial action against Gram-negative bacteria and Gram-positive bacteria.

An antimicrobial paint containing the water extract was prepared by the same method as that of the first embodiment, and an antimicrobial coating film was formed by using the antimicrobial paint. Antibacterial action of the antimicrobial coating film was evaluated by the same method as that of the first embodiment. Table 2 shows the evaluation test result of antibacterial action of the antimicrobial coating film of the present embodiment. Table 3 shows the evaluation test result of antibacterial action of an antimicrobial coating film containing the organic-solvent extract.

The result shows that an antimicrobial coating film containing a low MIC extract has antibacterial action. More specifically, the antimicrobial coating film containing a water extract of Kaemferia galanpal L. had antibacterial action against Gram-negative bacteria such as Escherichia coli. The antimicrobial coating film containing a water extract of Kaempferia galangal L. also had antibacterial action against Gram-positive bacteria such as Staphylococcus aureus.

On the other hand, an antimicrobial coating film containing an organic-solvent extract of Kaempferia galanpal L. did not have antibacterial action against Gram-negative bacteria and Gram-positive bacteria (not shown in the tables).

It was confirmed from the above result that the antimicrobial coating film of the present embodiment containing a water extract of a rhizome of Kaempferia galangal L. of family Zingiberaceae had antibacterial action against Gram-negative bacteria and Gram-positive bacteria.

### Third Embodiment

Respective antibacterial actions of the following water extracts, a method for producing antimicrobial coating films respectively containing the water extracts, and respective antibacterial actions of the antimicrobial coating films will be briefly described below: a water extract of a whole plant body of an aboveground portion of Isodon eriocalyx (Dunn) Kuds of family Laminaceae (Labiatae); a water extract of a rhizome of Scutellaria baicalensis Georgi of family Laminaceae (Labiatae); a water extract of a whole plant body of Usnea longissima Ach. of family Usneaceae; a water extract of bark, branches, and leaves of Pistacia weinmannifolia J. Poisson ex Franch of family Anacardiaceae; and a water extract of a whole plant body of Senecio scandens Buch.-Ham of family Compositae.

A whole plant body of an aboveground portion of Isodon eriocalyx (Dunn) Kuds of family Laminaceae (Labiatae), a rhizome of Scutellaria baicalensis Georgi of family Laminaceae (Labiatae), a whole plant body of Usnea longissima Ach. of family Usneaceae, bark, branches, and leaves of Pistacia weinmannifolia J. Poisson ex Franch of family Anacardiaceae, and a whole plant body of Senecio scandens Buch.-Ham of family Compositae were picked and dried in the shade in preparation for extraction.

A warm-water soluble material was obtained by water-extracting each plant body or a part thereof, more specifically, by steam distilling each plant body or a part thereof. Components of each warm-water soluble material were concentrated with a rotary evaporator. Each water extract has the following characteristics: a water extract of a whole plant body of an aboveground portion of Isodon eriocalyx (Dunn) Kuds of family Laminaceae (Labiatae) is a dark-brown sticky material; a water extract of a rhizome of Scutellaria baicalensis Georgi of family Laminaceae (Labiatae) is a dark-brown sticky material; a water extract of a whole plant body of Usnea longissima Ach. of family Usneaceae is a light-brown powdery material; a water extract of bark, branches, and leaves of Pistacia weinmannifolia J. Poisson ex Franch of family Anacardiaceae is a dark-brown sticky material; and a water extract of a whole plant body of Senecio scandens Buch.-Ham of family Compositae is a black, grease-like material.

Respective antibacterial actions of the water extracts and organic-solvent (acetone) extracts of the above plant bodies were evaluated. The result is shown in Table 1 for comparison. Note that species of bacteria, a method for evaluating antibacterial action, and an index (MIC) which are used in the third embodiment are the same as those used in the first embodiment. It is herein defined that a sample has antibacterial action when a MIC value is 1,600 or less (ppm).

The result shows that the water extracts of Isodon eriocalyx (Dunn) Kuds, Scutellaria baicalensis Georgi, Usnea longissima Ach., Pistacia weinmannifolia J. Poisson ex Franch, and Senecio scandens Buch.-Ham had antibacterial action against Gram-positive bacteria such as Staphylococcus aureus. On the other hand, the organic-solvent extracts of Isodon eriocalyx (Dunn) Kuds, Scutellaria baicalensis Georgi, Usnea longissima Ach., and Pistacia weinmannifolia J. Poisson ex Franch had antibacterial action against Gram-positive bacteria such as Staphylococcus aureus.

Antimicrobial paints respectively containing the above water extracts were prepared by the same method as that of the first embodiment, and antimicrobial coating films were formed by using the antimicrobial paints, respectively. Respective antibacterial actions of the antimicrobial coating films were evaluated by the same method as that of the first embodiment. Table 2 shows the evaluation test result of antibacterial action of the antimicrobial coating films of the present embodiment. Table 3 shows the evaluation test result of antibacterial action of antimicrobial coating films respectively containing the organic-solvent extracts.

The result shows that an antimicrobial coating film containing a low MIC extract has antibacterial action. More specifically, the antimicrobial coating films respectively containing the water extracts of Isodon eriocalyx (Dunn) Kuds, Scutellaria baicalensis Georgi, Usnea longissima Ach., Pistacia weinmannifolia J. Poisson ex Franch, and Senecio scandens Buch.-Ham had high antibacterial action against Gram-positive bacteria such as Staphylococcus aureus.

It was confirmed from the above result that the antimicrobial coating films of the present embodiment respectively containing water extracts of Isodon eriocalyx (Dunn) Kuds, Scutellaria baicalensis Georgi, Usnea longissima Ach., Pistacia weinmannifolia J. Poisson ex Franch, and Senecio scandens Buch.-Ham had high antibacterial action against Gram-positive bacteria. Note that a water extract of bark, branches, and leaves of Pistacia weinmannifolia J. Poisson ex Franch of family Anacardiaceae was used in the present embodiment. However, the level of antibacterial action obtained when one of a water extract of bark of Pistacia weinmannifolia J. Poisson ex Franch and a water extract of branches and leaves of Pistacia weinmannifolia J. Poisson ex Franch was used was the same as that obtained when the water extract of bark, branches, and leaves of Pistacia weinmannifolia J. Poisson ex Franch was used.

### Fourth Embodiment

Respective antibacterial actions of the following organic-solvent extracts, a method for producing antimicrobial coating films respectively containing the organic-solvent extracts, and respective antibacterial actions of the antimicrobial coating films will be briefly described below: an organic-solvent extract of a whole plant body of an aboveground portion of Isodon eriocalyx (Dunn) Kuds of family Laminaceae (Labiatae); an organic-solvent extract of a rhizome of Scutellaria baicalensis Georgi of family Laminaceae (Labiatae); an organic-solvent extract of a whole plant body of Usnea longissima Ach. of family Usneaceae; an organic-solvent extract of bark, branches, and leaves of Pistacia weinmannifolia J. Poisson ex Franch of family Anacardiaceae; and an organic-solvent extract of a rhizome of Rheum officinale Baill. of family Polygonaceae.

A whole plant body of an aboveground portion of Isodon eriocalyx (Dunn) Kuds of family Laminaceae (Labiatae), a rhizome of Scutellaria baicalensis Georgi of family Laminaceae (Labiatae), a whole plant body of Usnea longissima Ach. of family Usneaceae, bark, branches, and leaves of Pistacia weinmannifolia J. Poisson ex Franch of family Anacardiaceae, and a rhizome of Rheum officinale Baill. of family Polygonaceae were picked and dried in the shade in preparation for extraction.

Each plant body (a whole plant body or a part thereof) was extracted in an organic solvent, more specifically, in a mixed organic solvent of 70% acetone and 30% water. A material that is soluble in this organic solvent was obtained by distillation with a rotary evaporator. Components of the material were then concentrated with a rotary evaporator. Each organic-solvent extract has the following characteristics: an organic-solvent extract of a whole plant body of an aboveground portion of Isodon eriocalyx (Dunn) Kuds of family Laminaceae (Labiatae) is a dark earth-colored powdery material; an organic-solvent extract of a rhizome of Scutellaria baicalensis Georgi of family Laminaceae (Labiatae) is a yellow powdery material; an organic-solvent extract of a whole plant body of Usnea longissima Ach. of family Usneaceae is an ivory-colored, needle-crystal-like powdery material; an organic-solvent extract of bark, branches, and leaves of Pistacia weinmannifolia J. Poisson ex Franch of family Anacardiaceae is a light-orange powdery material; and an organic-solvent extract of a rhizome of Rheum officinale Baill. of family Polygonaceae is a dark-brown, highly sticky material.

Respective antibacterial actions of the organic-solvent extracts and water extracts of the above plant bodies were evaluated. The result is shown in Table 1 for comparison. Note that species of bacteria, a method for evaluating antibacterial action, and an index (MIC) which are used in the fourth embodiment are the same as those used in the first embodiment. It is herein defined that a sample has antibacterial action when a MIC value is 1,600 or less (ppm).

The result shows that the water extracts of Isodon eriocalyx (Dunn) Kuds, Scutellaria baicalensis Georgi, Usnea longissima Ach., and Pistacia weinmannifolia J. Poisson ex Franch had antibacterial action against Gram-positive bacteria such as Staphylococcus aureus. On the other hand, the organic-solvent extracts of Isodon eriocalyx (Dunn) Kuds, Scutellaria baicalensis Georgi, Usnea longissima Ach., Pistacia weinmannifolia J. Poisson ex Franch, and Rheum officinale Baill. had antibacterial action against Gram-positive bacteria such as Staphylococcus aureus.

Hereinafter, a method for producing antimicrobial paints respectively containing the organic-solvent extracts of the above plant bodies and a method for producing antimicrobial coating films respectively containing the organic-solvent extracts of the above plant bodies will be described. First, a mixed solution of 10 parts by weight of a water-based urethane resin (based on solid weight) and 1 part by weight of an acrylic latex (based on solid weight) was prepared as a paint binder (paint resin). 1 part by weight of the organic-solvent extract was mixed with the mixed solution and well dispersed therein. An antimicrobial composition was thus prepared. RA85 (trade name) produced by Nippon NSC Ltd. was used as a water-based urethane resin and LX854C (trade name) produced by Zeon Corporation was used as an acrylic latex.

The antimicrobial paints respectively containing the organic-solvent extracts and the antimicrobial coating films respectively containing the organic-solvent extracts were produced by the same method as that of the first embodiment. Respective antibacterial actions of the antimicrobial coating films were evaluated by the same method as that of the first embodiment. Table 3 shows the evaluation test result of antibacterial action of the antimicrobial coating films of the present embodiment.

The result shows that an antimicrobial coating film containing a low MIC extract has high antibacterial action. More specifically, the antimicrobial coating films respectively containing the organic-solvent extracts of Isodon eriocalyx (Dunn) Kuds, Scutellaria baicalensis Georgi, Usnea longissima Ach., Pistacia weinmannifolia J. Poisson ex Franch, and Rheum officinale Baill. had high antibacterial action against Gram-positive bacteria such as Staphylococcus aureus. However, none of the antibacterial coating films had high antibacterial action against Gram-negative bacteria such as Escherichia coli.

It was confirmed from the above result that the antimicrobial coating films of the present embodiment respectively containing the organic-solvent extracts of Isodon eriocalyx (Dunn) Kuds, Scutellaria baicalensis Georgi, Usnea longissima Ach., Pistacia weinmannifolia J. Poisson ex Franch, and Rheum officinale Baill. had high. antibacterial action against Gram-positive bacteria. Note that an organic-solvent extract of bark, branches, and leaves of Pistacia weinmannifolia J. Poisson ex Franch of family Anacardiaceae was used in the present embodiment. However, the level of antibacterial action obtained when one of an organic-solvent extract of bark of Pistacia weinmannifolia J. Poisson ex Franch and an organic-solvent extract of branches and leaves of Pistacia weinmannifolia J. Poisson ex Franch was used was the same as that obtained when the organic-solvent extract of bark, branches, and leaves of Pistacia weinmannifolia J. Poisson ex Franch was used.

### Fifth Embodiment

In the present embodiment, influences of an extract concentration of a coating film on antibacterial action and coating-film characteristics will be evaluated. The organic-solvent extract of Usnea longissima Ach. prepared in the fourth embodiment was used for the test. A water-soluble urethane resin was used with water as a solvent. 0.1 to 500 parts by weight of the organic-solvent extract was mixed with 100 parts by weight of the water-soluble urethane resin based on solid weight and dispersed therein. Antibacterial action of the resultant material was evaluated. The evaluation test method was the same as that used in the first embodiment. Samples used for evaluation test were produced as follows:

(1) An antimicrobial paint was prepared by mixing 1 part by weight of the organic-solvent extract, 100 parts by weight of the resin (based on solid weight), and a coating solvent (water). The mixture was diluted with the coating solvent (water) to an easy-to-coat viscosity.

(2) The paint prepared in (1) was applied to the surface of a 5 cm square PET film of 125 μm thick by a roll coater method at about 100 mg/25 cm² (based on dry weight). The resultant PET film was sufficiently dried in a dust-free place with good ventilation. The PET film was then forced-dried with a drier at 60° C. for about one hour. Antibacterial action of the PET film was then evaluated.

“Blank” samples, samples that do not contain the organic-solvent extract but contain other bark components, were produced by the same method. Each sample was weighed before and after the coating process in order to obtain the net weight of the organic-solvent extract. Similarly, samples having various mixing ratios of the organic-solvent extract and the paint resin were produced and antibacterial action of each sample was evaluated. The result is shown in Table 4. In Table 4, “⊚” indicates that the ratio of the survival cell count (blank/sample) after 24 hours from the initiation of the test was 1,000 or more, “◯” indicates that this ratio was less than 1,000 and 100 or more, and “Δ” indicates that this ratio was less than 100 and 50 or more.

TABLE 4

| The mixing amount of | | |
|---|---|---|
| the extract (based | Antibacterial | Coating- |
| on solid weight) | action | film characteristics |
| 0.1 | Δ | Firm coating film |
| 0.5 | ◯ | Firm coating film |
| 1.0 | ⊚ | Firm coating film |
| 2 | ⊚ | Firm coating film |
| 5 | ⊚ | Firm coating film |
| 10 | ⊚ | Firm coating film |
| 50 | ⊚ | Firm coating film |
| 100 | ⊚ | Good coating film |
| 500 | ⊚ | Poor adhesion to a base material |

The results shows that a coating film has practical or higher antibacterial action against microorganisms that are present in the living environment when the antimicrobial paint is applied at a dry weight ratio of 0.1 or more in the coating film. In particular, it was confirmed that an antimicrobial coating film has practical antibacterial action when the antimicrobial coating film is formed with an antimicrobial paint formed by mixing 1 to 500 parts by weight of the organic-solvent extract with 100 parts by weight of the water-soluble urethane resin. It was also confirmed that practical coating-film characteristics is obtained when 100 or less parts by weight of the organic-solvent extract is mixed with 100 parts by weight of the water-soluble urethane resin. The antimicrobial coating film is considered to be highly safe because the ingredients of the extract have been used as ingredients of Chinese herb medicines for a long time.

The same tendencies were obtained even when polyvinyl alcohol, carboxymethylcellulose, epoxy resin, or polyester resin was used as a paint resin instead of water-soluble urethane resin. The same experimentation was conducted for other extracts and the same results were obtained.

### Sixth Embodiment

An antimicrobial paint having active ingredients of a plant body held by an inorganic carrier, and an antimicrobial coating film formed using the antimicrobial paint will be described in the present embodiment. Bark and/or branches and leaves of Pistacia weinmannifolia J. Poisson ex Franch of family Anacardiaceae were used as a plant body. This plant body was sufficiently dried in preparation for extraction; When the extraction process is conducted before the plant body is sufficiently dried, an extract quality may be degraded (e.g., gelling of extracted ingredients). This results in instability of the extract quality.

Bark and/or branches and leaves of Pistacia weinmannifolia J. Poisson ex Franch of family Anacardiaceae were immersed in water. As a result, a warm-water soluble material was obtained. The extracted material was powdered by a spray dry method. Powder of a hydrotalcite compound (powder produced by Toda Kogyo Corp.) was prepared as an inorganic carrier (plate face diameter: 0.3 μm; thickness: 0.06 μm; and specific surface area: 14 m²/g). 1 part by weight of the powder of the hydrotalcite compound and 1 part by weight of the extract were dispersed in a mixed solvent of 70% ethyl alcohol and 30% water. After the powder and the extract were well dispersed in the mixed solvent, the solvent was vaporized while stirring. Antimicrobial particles having the extract held by the inorganic carrier were thus obtained.

A water paint was prepared by dissolving polyvinyl alcohol (a paint resin) in water, and an antimicrobial paint was prepared by dispersing the antimicrobial particles in the water paint. More specifically, this antimicrobial paint was prepared so that the antimicrobial paint contains 5 parts by weight of the extract, 5 parts by weight of the hydrotalcite compound (inorganic carrier), and 90 parts by weight of polyvinyl alcohol, based on the weight of a coating film of the antimicrobial paint. An antimicrobial coating film was produced by using this antimicrobial paint. Antibacterial action of this antimicrobial coating film was evaluated by the evaluation method of the first embodiment. It was confirmed from the result that the antimicrobial coating film in which antimicrobial particles holding the extract are dispersed has practical coating-film characteristics and practical antibacterial action against Gram-positive bacteria.

Since the extract is held by the inorganic carrier in the present embodiment, heat resistance of the extract can be improved. Moreover, the extract is present not only on the outer surface of the inorganic carrier but within pores of the inorganic carrier. Therefore, even when the extract on the outer surface is lost due to rain or the like, antimicrobial action can be maintained by the extract remaining in the pores.

### Seventh Embodiment

An antimicrobial paint and an antimicrobial coating film were produced in the same manner as that of the fifth embodiment except that a whole plant body of an aboveground portion of Isodon eriocalyx (Dunn) Kuds of family Laminaceae (Labiatae) was used as a plant body. A whole plant body of an aboveground portion of Isodon eriocalyx (Dunn) Kuds of family Laminaceae (Labiatae) was cut off from summer to fall. Foreign matter was then removed and the whole plant body of the aboveground portion of Isodon eriocalyx (Dunn) Kuds was sufficiently dried in preparation for extraction.

The whole plant body of the aboveground portion of Isodon eriocalyx (Dunn) Kuds was immersed in a mixed organic solvent of 70% acetone and 30% water, and a material that is soluble in a mixed solvent was extracted. Components of the material were concentrated by a spray dry method. As a result, dark earth-colored powder that is soluble in a water/ethanol solvent was obtained.

Powder of a hydrotalcite compound which is shown in the sixth embodiment was prepared as an inorganic carrier. 1 part by weight of the powder of the hydrotalcite compound and 1 part by weight of the extract were dispersed in a mixed solvent of 70% ethyl alcohol and 30% water. After the powder and the extract were well dispersed in the mixed solvent, the solvent was vaporized while stirring. Antimicrobial particles having the extract held by the inorganic carrier were thus obtained.

As in the sixth embodiment, a water paint was prepared by dissolving polyvinyl alcohol (a paint resin) in water (a coating solvent), and an antimicrobial paint was prepared by dispersing the antimicrobial particles in the water paint. Antibacterial action of this antimicrobial paint was evaluated by the evaluation method of the first embodiment. It was confirmed from the result that an antimicrobial coating film in which antimicrobial particles holding the extract are dispersed has practical coating-film characteristics and practical antibacterial action against Gram-positive bacteria.

The same tendencies were obtained even when a water-soluble urethane resin, carboxymethylcellulose, epoxy resin, or polyester resin was used as a paint resin instead of polyvinyl alcohol. The same experimentation was conducted for other extracts and the same results were obtained.

### Eighth Embodiment

Antimicrobial coating films were respectively prepared in the same manner as that of the sixth and seventh embodiments except that particulate powder was used as an inorganic carrier. The particulate powder used in the present embodiment is formed mainly from a porous inorganic oxide, has a specific surface area of 300 m²/g or more, has silanol groups at its surface, and has a grain size of 50 μm or less. More specifically, antimicrobial particles were prepared by the same process as that of the sixth and seventh embodiments by using fine silica gel particles treated to have a hydrophobic surface and having a grain size of 1 μm (trade name: Sylophobic 200, a product of Fuji Silysia Chemical Corp.).

Antimicrobial paints were prepared by using the antimicrobial particles. More specifically, antimicrobial paints were prepared by mixing 5 parts by weight of a water extract of bark and/or branches and leaves of Pistacia weinmannifolia J. Poisson ex Franch of family Anacardiaceae (or an organic-solvent extract of a whole plant body of an aboveground portion of Isodon eriocalyx (Dunn) Kuds of family Laminaceae (Labiatae)), 2 parts by weight of silica gel powder, and 93 parts by weight of a water-soluble urethane resin (based on solid weight).

It was confirmed that, as in the case where a hydrotalcite compound was used as an inorganic carrier, the antimicrobial coating films that contain fine silica gel particles holding the extract have practical coating-film characteristics and practical antibacterial action.

The same tendencies were obtained even when polyvinyl alcohol, carboxymethylcellulose, epoxy resin, or polyester resin was used as a paint resin instead of water-soluble urethane resin. The same experimentation was conducted for other extracts and the same results were obtained.

### Ninth Embodiment

Catechin has recently drawn attention for its various effects. In the present embodiment, an antimicrobial paint containing catechin and at least one kind of plant body extract and an antimicrobial coating film formed by using the antimicrobial paint will be described. The antimicrobial paint and the antimicrobial coating film of the present embodiment contain a water extract of branches and leaves of a plant of Eucalyptus globulus Labill. of family Myrtaceae, catechin, and a coating solvent.

Catechin is water-soluble components contained in tea leaves. Catechin is commonly called tannin due to its bitter taste. There are various kinds of tea catechin such as epigallocatechingallate, epicatechin, epicatechingallate, epigallocatechin, and catechin.

Catechin that is used in the present embodiment is an extract of tea leaves and is reported to have an MIC value of 250 ppm to 800 ppm against Staphylococcus aureus (Gram-positive bacteria) (Kokinzai no Kagaku II (Japanese title) (Science of Antibacterial Agents II), published by Kogyo Chosakai Publishing Inc.) However, antibacterial action of catechin against Gram-negative bacteria has not been reported.

A water extract of branches and leaves of Eucalyptus globulus Labill. of family Myrtaceae from China was prepared by the method described in the first embodiment. An antimicrobial paint was prepared by mixing the water extract and catechin at a weight ratio of 1:1 and dispersing the mixture in a coating solvent (water) at such a concentration that provides coating ability. A spray container is filled with this antimicrobial paint, and an antimicrobial coating film was produced by spraying the antimicrobial paint on a sheet of paper. Antibacterial action of the paper was then evaluated. It was confirmed from the result that the antimicrobial coating film of the present embodiment has high antibacterial action against Gram-positive bacteria and Gram-negative bacteria. In other words, the paper was sufficiently antibacteria-treated.

As in the fifth embodiment, an antimicrobial coating film was produced by applying this antimicrobial paint to a surface of a PET film by a roll coater method. Antibacterial action of this antimicrobial coating film was then evaluated. It was confirmed from the result that the antimicrobial coating film has practical antibacterial action and practical coating-film characteristics.

### Tenth Embodiment

An antimicrobial paint containing a silver-based antibacterial agent and an antimicrobial coating film that is produced by using the antimicrobial paint will be described in the present embodiment. The antimicrobial paint and the antimicrobial coating film of the present embodiment contain a water extract of a rhizome of Scutellaria baicalensis Georgi of family Laminaceae (Labiatae), a silver-complex based antibacterial agent, a paint resin, and a coating solvent.

A rhizome of Scutellaria baicalensis Georgi of family Laminaceae (Labiatae) was sufficiently dried in preparation for extraction. A warm-water soluble material was obtained by immersing the rhizome of Scutellaria baicalensis Georgi in water. Yellow powder was obtained by pulverizing this material by a spray dry method.

A silver-complex based antibacterial agent used in the present embodiment is silica gel having thiosulphato silver complexes adsorbed therein and having its surface covered with a tetraethoxysilane hydrolyzate. 1 part by weight of the silver-complex based antibacterial agent (trade name: Amenitop, a product of Matsushita Electric Industrial Co., Ltd.) and 1 part by weight of the water extract of a rhizome of Scutellaria baicalensis Georgi were dispersed in 20 parts by weight of water as a solvent. The resultant solution was dried by vaporization while stirring. Antibacterial particles, i.e., the silver-complex based antibacterial agent with the water extract adhering to its surface, were thus prepared. A paint binder was prepared by dispersing 98 parts by weight of carboxymethylcellulose in a coating solvent (water) to such a degree that provides coating ability. An antimicrobial paint was prepared by dispersing 2 parts by weight of the above antibacterial particles in the paint binder.

An antimicrobial coating film was produced by the same method as that of the fifth embodiment by using this antimicrobial paint, and antibacterial action of the antimicrobial coating film was evaluated. It was confirmed from the result that the antimicrobial coating film has practical antibacterial action and practical coating-film characteristics.

The same tendencies were obtained even when polyvinyl alcohol, water-soluble urethane resin, epoxy resin, or polyester resin was used as a paint resin instead of carboxymethylcellulose. The same experimentation was conducted for other extracts and the same results were obtained.

### Eleventh Embodiment

An antimicrobial paint containing a plurality of kinds of plant body extracts, catechin, and a silver-based antibacterial agent and an antimicrobial coating film that is formed by using the antimicrobial paint will be described in the present embodiment. The antimicrobial paint and the antimicrobial coating film of the present embodiment contain a water extract of a rhizome of Kaemferia galangal L. of family Zingiberaceae, a water extract of a whole plant body of Usnea longissima Ach. of family Usneaceae, catechine, a silver-complex based antibacterial agent, a paint resin, and a coating solvent.

A water extract of a rhizome of Kaempferia galangal L. of family Zingiberaceae was prepared in the same manner as that of the second embodiment. A water extract of a whole plant body of Usnea longissima Ach. of family Usneaceae was prepared in the same manner as that of the third embodiment. 1 part by weight of a silver-complex based antibacterial agent (trade name: Amenitop, a product of Matsushita Electric Industrial Co., Ltd.), 1 part by weight of the water extract of a rhizome of Kaempferia galangal L., 1 part by weight of the water extract of a whole plant body of Usnea longissima Ach., and 1 part by weight of catechin were dispersed in 20 parts by weight of water as a solvent. The resultant solution was dried by vaporization while stirring. Antibacterial particles, i.e., the silver-complex based antibacterial agent with the two kinds of water extracts and catechin adhering to its surface, were thus prepared. An antimicrobial paint was then prepared in the same manner as that of the tenth embodiment.

An antimicrobial coating film was produced by the same method as that of the first embodiment by using this antimicrobial paint, and antibacterial action of the antimicrobial coating film was evaluated. It was confirmed from the result that the antimicrobial coating film of the present embodiment has practical antibacterial action and practical coating-film characteristics.

### Twelfth Embodiment

An antimicrobial paint was prepared by the same method as that of the eleventh embodiment except that the two kinds of water extracts used in the eleventh embodiment were replaced with two kinds of organic-solvent extracts. Two of the organic-solvent extracts prepared in the fourth embodiments were used in the present embodiment. More specifically, an organic-solvent extract of a whole plant body of an aboveground portion of Isodon eriocalyx (Dunn) Kuds of family Laminaceae (Labiatae) and an organic-solvent extract of a whole plant body of Usnea longissima Ach. of family Usneaceae were used in the twelfth embodiment.

An antimicrobial coating film was produced by the same method as that of the first embodiment by using an antimicrobial paint of the present embodiment, and antibacterial action of the antimicrobial coating film was evaluated. It was confirmed from the result that the antimicrobial coating film of the present embodiment has practical antibacterial action and practical coating-film characteristics.

The eleventh and twelfth embodiments show a paint and a coating film which contain a plurality of water extracts or a plurality of organic-solvent extracts of plant bodies. However, a paint and a coating film of the eleventh and twelfth embodiments may include both at least one water extract and at least one organic-solvent extract of a plant body.

### Thirteenth Embodiment

An air filter having an antimicrobial coating film will be described in the present embodiment. A water extract of branches and leaves of Eucalyptus globulus Labill. of family Myrtaceae from China was prepared by the same method as that of the first embodiment. A water extract of a whole plant body of Usnea lonpissima Ach. of family Usneaceae was prepared by the same method as that of the fourth embodiment. The two water extracts thus prepared were mixed at a mixing ratio of 1:1. 2 parts by weight of this extract mixture was mixed with 98 parts by weight of aqueous acrylic resin (based on solid weight) and dispersed therein. An antimicrobial paint was thus prepared.

Non-woven fabric for air-cleaner filters was prepared. This filter includes a pre-filter layer and a charged filter layer. The pre-filter layer is polypropylene non-woven fabric formed from fibers of about 30 μm in diameter. The charged-filter layer is formed from ultra-thin polypropylene fibers meltblown on the pre-filter layer. The antimicrobial paint was printed only on the surface of the pre-filter layer which is located on the opposite side of the charged filter layer by a gravure method. An antimicrobial coating film was formed by drying the printed antimicrobial paint.

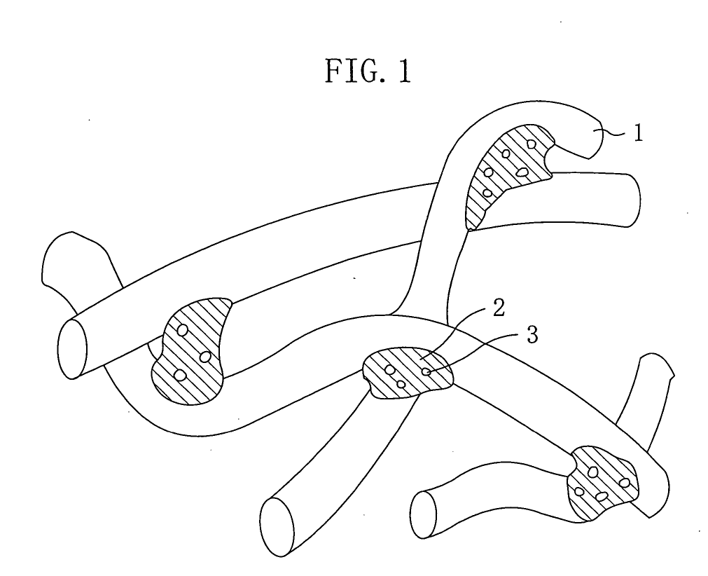

FIG. 1 is a partial enlarged view of the pre-filter layer. As shown in FIG. 1, non-woven fabric has a plurality of polypropylene fibers 1 that cross each other. An antimicrobial coating film 2 with an extract mixture 3 dispersed therein is formed at intersections of the fibers 1. Note that, in fact, the extract mixture 3 has been dissolved and dispersed in the antimicrobial coating film 2 and the extract mixture 3 is not in the form of particles. In FIG. 1, the extract mixture 3 is shown in the form of particles in order to show dispersion schematically.

By using a gravure method, the antimicrobial paint can be applied and painted to at least intersections of fibers of the non-woven fabric. In other words, the antimicrobial paint is applied near the intersections of fibers at the surface of the pre-filter layer. Therefore, the antimicrobial paint does not get into the filter layer, and pressure loss of the air passing through the filter is not likely to occur. The antimicrobial coating film 2 has antibacterial action against microorganisms that are captured from the air stream on the surface of the filter portion (especially near the intersections of fibers).

Table 5 shows the result of antibacterial action of the filter surface. Microorganisms activated in the logarithmic growth phase (initial cell count) were dropped on the filter, and the survival cell count was measured after the filter was let stand in an atmosphere of 35° C. for 24 hours. Antibacterial action of a conventional filter that has not been coated with an antimicrobial paint was also evaluated in the same manner. The result is shown as “conventional filter” in Table 5. The numerals in Table 5 indicate the viable cell count on the filter surface (cfu/ml (colony forming units per milliliter)).

TABLE 5

Survival cell count
after 24 hours

| Evaluated | Initial | Present | Conventional |
|---|---|---|---|
| microorganisms | cell count | invention | filter |
| Escherichia coli | 1.0 × 10⁵ | 0 | 5.6 × 10⁴ |
| Staphylococcus aureus | 1.2 × 10⁵ | 0 | 8.0 × 10⁴ |
| MRSA | 2.0 × 10⁵ | 0 | 1.0 × 10⁵ |

Table 5 shows that the filter of the present embodiment had practical antibacterial action. Moreover, an air cleaner having the filter of the present embodiment attached action. Moreover, an air cleaner having the filter of the present embodiment attached thereto also had practical antibacterial action.

Although filters for air cleaner are described in the present embodiment, a filter having the antimicrobial coating film may be used for water purifiers.

### Fourteenth Embodiment

In the present embodiment, a medical chart file is described as an example of an industrial product having an antimicrobial coating film. A water extract of branches and leaves of Eucalyptus globulus Labill. of family Myrtaceae from China was prepared by the same method as that of the first embodiment. A water extract of a rhizome of Kaempferia galangal L. of family Zingiberaceae was prepared by the same method as that of the second embodiment. The two water extracts thus prepared were mixed at a mixing ratio of 1:1. 3 parts by weight of this extract mixture is mixed with 97 parts by weight of aqueous acrylic resin (based on solid weight) and dispersed therein. An antimicrobial paint was thus prepared.

A sheet of paper (150 g/m²) was prepared as a base material. The paper was impregnated with synthetic rubber latex resin. A film of soft vinyl chloride resin of about 80 μm thick was formed on the paper surface by a calendering process in order to adjust texture and color tone of the surface. The surface-treated paper was coated with the above antimicrobial paint with a thickness of about 2 μm to about 6 μm by a gravure coating method. The resultant paper was cut, punched, and folded. A medical chart file was thus produced.

Antibacterial action of the surface of the medical chart file was evaluated by the same test method as that of the first embodiment. The result is shown in Table 6. Antibacterial action of a medical chart file with a non-antibacterial-treated surface was also evaluated by the same method. The result is shown as “non-treated product” in Table 6. medical chart files.

TABLE 6

| | | Escherichia | Staphylococcus |
|---|---|---|---|
| | | coli | aureus |
| Initial cell count | | 5.3 × 10⁴ | 4.5 × 10⁴ |
| Survival cell count | Non-treated | >10⁶ | 2.8 × 10⁴ |
| after 24 hours | product | | |
| | Present | <10 | <10 |
| | invention | | |

Table 6 shows that the medical chart file of the present embodiment has practical antibacterial action. Industrial products having an antimicrobial coating film were produced by coating a housing or a base material of a part with an antimicrobial paint containing at least one kind of plant body extracts defined in the present invention or by impregnating a base material of a part with the antimicrobial paint. The industrial products thus produced are wallpapers, stationery, auto parts, furniture, cooking equipments, and medical equipments. The surfaces of these industrial products had practical antibacterial action.

Although the embodiments of the present invention have been described, the technical scope of the present invention is not limited to that described in the above embodiments. It should be understood by those skilled in the art that the above embodiments are exemplary only, and that various modifications in which the components of the above embodiments are modified in various ways are possible and such modifications also fall within the technical scope of the present invention.

The composition and the antimicrobial coating film of the present invention have strong antimicrobial action against harmful microorganisms that are present in the living environment, that is, microorganisms that should be gotten rid of to create amenity environments. Moreover, the plant body extracts that are used in the present invention have been confirmed to be safe because they have been used as ingredients of Chinese herb medicines for a long time. Materials in the living environment (fixtures and fittings, medicines for a long time. Materials in the living environment (fixtures and fittings, construction materials, and the like) which are coated with a composition of the present invention or which have a coating film of the present invention can be used without anxiety due to their high safety. Unlike synthetic antibacterial agents that are used only in emergency situations, this effect is necessary to place the composition and the coating film of the present invention in the living environment.

Accordingly, by using the composition of the present invention for antimicrobial treatment of materials in the living environment or by forming the antimicrobial coating film of the present invention on materials in the living environment, the living environment can be kept clean and living-related materials such as construction materials can be prevented from being degraded by microorganisms with high safety. Moreover, by suppressing proliferation of harmful microorganisms, allergies can be prevented from being caused by floating microorganisms. The present invention thus has great industrial effects.

The antimicrobial paint and the antimicrobial coating film of the present invention can be used to prevent various harmful effects of microorganisms. For example, the antimicrobial paint and the antimicrobial coating film of the present invention are useful for applications such as antibacterial surface treatment of living-related materials, preservative treatment of industrial products and members thereof, treatment for preventing corrosion of construction-related materials by microorganisms, preservative treatment of food-related materials, microbiological control and odor control for industrial wrapping materials, and microbiological control in water treatment. The antimicrobial paint and the antimicrobial coating film of the present invention are also applicable as a microbiological control agent for medical and pharmaceutical materials, a disinfectant for public hygiene, and the like.

What is claimed is:

1. A coating-type antimicrobial composition containing at least: a water extract of at least one kind of plant body selected from the group consisting of branches and leaves of a plant of family Myrtaceae, genus Eucalyptus, a rhizome of a plant of family Zingiberaceae, genus Kaempferia, a whole plant body of an aboveground portion of a plant of family Laminaceae (Labiatae), genus Isodon, a rhizome of a plant of family Laminaceae (Labiatae), genus Scutellaria, a whole plant body of a plant of family Usneaceae, genus Usnea, bark, branches and leaves of a plant of family Anacardiaceae, genus Pistacia, and a whole plant body of a plant of family Compositae, genus Senecio; and a paint resin and/or a coating solvent.

2. The coating-type antimicrobial composition according to claim 1, further containing an inorganic carrier.

3. The coating-type antimicrobial composition according to claim 1, further containing catechin and/or a silver-based antibacterial agent.

4. The coating-type antimicrobial composition according to claim 3, wherein the silver-based antibacterial agent includes a carrier holding silver complexes and/or silver ions.

5. A coating-type antimicrobial composition containing at least: an organic-solvent extract of at least one kind of plant body selected from the group consisting of a whole plant body of an aboveground portion of a plant of family Laminaceae (Labiatae), genus Isodon, a rhizome of a plant of family Laminaceae (Labiatae), genus Scutellaria, a whole plant body of a plant of family Usneaceae, genus Usnea, bark, branches and leaves of a plant of family Anacardiaceae, genus Pistacia, and a rhizome of a plant of family Polygonaceae, genus Rheum; and a paint resin and/or a coating solvent.

6. The coating-type antimicrobial composition according to claim 5, further containing an inorganic carrier.

7. The coating-type antimicrobial composition according to claim 5, further containing catechin and/or a silver-based antibacterial agent.

8. The coating-type antimicrobial composition according to claim 7, wherein the silver-based antibacterial agent includes a carrier holding silver complexes and/or silver ions.

9. An antimicrobial coating film containing a water extract of at least one kind of plant body selected from the group consisting of: branches and leaves of a plant of family Myrtaceae, genus Eucalyptus; a rhizome of a plant of family Zingiberaceae, genus Kaempferia; a whole plant body of an aboveground portion of a plant of family Laminaceae (Labiatae), genus Isodon; a rhizome of a plant of family Laminaceae (Labiatae), genus Scutellaria; a whole plant body of a plant of family Usneaceae, genus Usnea; bark, branches and leaves of a plant of family Anacardiaceae, genus Pistacia; and a whole plant body of a plant of family Compositae, genus Senecio.

10. The antimicrobial coating film according to claim 9, further containing a paint resin.

11. The antimicrobial coating film according to claim 10, wherein the paint resin contains a resin contained in a water paint, a resin emulsified and dispersed in water.

12. The antimicrobial coating film according to claim 10, wherein the antimicrobial coating film contains 1 to 500 parts by weight of the water extract for 100 parts by weight of a solid matter of the paint resin.

13. The antimicrobial coating film according to claim 9, further containing an inorganic carrier.

14. The antimicrobial coating film according to claim 13, wherein the inorganic carrier is particulate powder formed from an intercalation compound.

15. The antimicrobial coating film according to claim 13, wherein the inorganic carrier is particulate powder formed mainly from a porous inorganic oxide, having a specific surface area of at least 300 m²/g, having silanol groups at a surface, and having a grain size of at most 50 μm.

16. The antimicrobial coating film according to claim 9, further containing catechin and/or a silver-based antibacterial agent.

17. The antimicrobial coating film according to claim 16, wherein the silver-based antibacterial agent includes a carrier holding silver complexes and/or silver ions.

18. A filter formed from a plurality of fibers crossing each other, wherein the antimicrobial coating film of claim 9 is formed at intersections of the fibers.

19. An electric air-quality conditioning equipment having the filter of claim 18 attached thereto.

20. The electric air-quality conditioning equipment according to claim 19 which is any one of an air cleaner, an air conditioner, a humidifier, and a vacuum cleaner.

21. An antimicrobial coating film containing an organic-solvent extract of at least one kind. of plant body selected from the group consisting of: a whole plant body of an aboveground portion of a plant of family Laminaceae (Labiatae), genus Isodon; a rhizome of a plant of family Laminaceae (Labiatae), genus Scutellaria; a whole plant body of a plant of family Usneaceae, genus Usnea; bark, branches and leaves of a plant of family Anacardiaceae, genus Pistacia; and a rhizome of a plant of family Polygonaceae, genus Rheum.

22. The antimicrobial coating film according to claim 21, further containing a paint resin.

23. The antimicrobial coating film according to claim 22, the paint resin contains a resin contained in a water paint, a resin emulsified and dispersed in water.

24. The antimicrobial coating film according to claim 22, wherein the antimicrobial coating film contains 1 to 500 parts by weight of the organic-solvent extract for 100 parts by weight of a solid matter of the paint resin.

25. The antimicrobial coating film according to claim 21, further containing an inorganic carrier.

26. The antimicrobial coating film according to claim 25, wherein the inorganic carrier is particulate powder formed from an intercalation compound.

27. The antimicrobial coating film according to claim 25, wherein the inorganic carrier is particulate powder formed mainly from a porous inorganic oxide, having a specific surface area of at least 300 m²/g, having silanol groups at a surface, and having a grain size of at most 50 μm.

28. The antimicrobial coating film according to claim 21, further containing catechin and/or a silver-based antibacterial agent.

29. The antimicrobial coating film according to claim 28, wherein the silver-based antibacterial agent includes a carrier holding silver complexes and/or silver ions.

30. A filter formed from a plurality of fibers crossing each other, wherein the antimicrobial coating film of claim 21 is formed at intersections of the fibers.

31. An electric air-quality conditioning equipment having the filter of claim 30 attached thereto.

32. The electric air-quality conditioning equipment according to claim 31 which is any one of an air cleaner, an air conditioner, a humidifier, and a vacuum cleaner.
